(19) United States
(12) Patent Application Publication
(10) Pub. No.: US 2022/0084199 A1
(43) Pub. Date: Mar. 17, 2022

(54) METHOD FOR ANALYZING BIOMATERIALS BASED ON IMAGE ANALYSIS AND PERSONALIZATION DATA

(71) Applicant: Samsung Electronics Co., Ltd., Suwon-si (KR)

(72) Inventors: Sukyong LEE, Suwon-si (KR); Seokho KIM, Suwon-si (KR); Seunghyun KIM, Suwon-si (KR); Hangchan KIM, Suwon-si (KR); Sanghyun LEE, Suwon-si (KR)

(21) Appl. No.: 17/469,325

(22) Filed: Sep. 8, 2021

(30) Foreign Application Priority Data
Sep. 11, 2020 (KR) 10-2020-0116948

(51) Int. Cl. G06T 7/00; A61B 5/00; G01N 33/52

(52) U.S. Cl. CPC G06T 7/0012; G06T 2207/10024; G01N 33/52; A61B 5/0013

(57) ABSTRACT

An electronic device and a control method of an electronic device are provided. The control method includes obtaining an image on the diagnostic kit through a camera included in the electronic device, the obtaining of the image based on receiving a user command for photographing a diagnostic kit including at least one test line discolored according to a reaction between a sample collected from a body of a user and a reagent, and a plurality of tone distinction markers with tones different from one another, obtaining a first color value on the at least one test line included in the obtained image and a second color value on each of the plurality of tone distinction markers, obtaining a first deviation information on a photographing environment of the obtained image, correcting a first data to obtain a biometric information measurement value, and obtaining a diagnostic result corresponding to the measurement value.

### CROSS-REFERENCE TO RELATED APPLICATION(S)

This application is based on and claims priority under 35 U.S.C. § 119(a) of a Korean patent application number 10-2020-0116948, filed on Sep. 11, 2020, in the Korean Intellectual Property Office, the disclosure of which is incorporated by reference herein in its entirety.

### BACKGROUND

### 1. Field

The disclosure relates to an electronic device and a control method thereof. More particularly, the disclosure relates to an electronic device capable of obtaining quantitative values on biometric information collected from a body of a user by analyzing an image on a diagnostic kit and a control method of an electronic device.

### 2. Description of Related Art

Recently, technology of measuring biometric information based on samples collected from a body of a user are being developed continuously. In particular, research on technology which obtains an image of a diagnostic kit, in which a sample is collected through a camera of an electronic device, and provides a more accurate biometric information using an easy method to a user by analyzing the image, is actively underway.

However, in the process of obtaining an image on the diagnostic kit through the camera of the electronic device, there may be a difference between a color of a test line of an actual diagnostic kit and a color of a test line included in the image. Accordingly, if biometric information is obtained simply based on the color itself of the test line included in the image, there may be the problem of providing inaccurate biometric information to the user.

Accordingly, by taking into consideration the various photographing environments such as a lighting of space the diagnostic kit is photographed in, a manufacturing deviation generated in the manufacturing process of the corresponding diagnostic kit, intrinsic characteristics on biometric information for each user, and the like, there is a growing need for technology which can provide a more accurate biometric information.

The above information is presented as background information only to assist with an understanding of the disclosure. No determination has been made, and no assertion is made, as to whether any of the above might be applicable as prior art with regard to the disclosure.

### SUMMARY

Aspects of the disclosure are to address at least the above mentioned problems and/or disadvantages and to provide at least the advantages described below. Accordingly, an aspect of the disclosure is to provide an electronic device for providing a diagnostic result by more accurately analyzing a quantitative value on a color of a test line apparent in a diagnostic kit and a control method thereof.

Additional aspects will be set forth in part in the description which follows and, in part, will be apparent from the description, or may be learned by practice of the presented embodiments.

In accordance with an aspect of the disclosure, an electronic device is provided. The electronic device includes a camera, a memory configured to store a first data to obtain a biometric information measurement value according to a color value and a second data to obtain a diagnostic result according to the biometric information measurement value, and a processor configured to obtain, based on a user command for photographing a diagnostic kit including at least one test line discolored according to a reaction between a sample collected from a body of a user and a reagent and a plurality of tone distinction markers with different tones from one another, an image on the diagnostic kit through the camera, obtain a first color value on the at least one test line included in the obtained image and a second color value on each of the plurality of tone distinction markers included in the obtained image, obtain a first deviation information on a photographing environment of the obtained image based on the second color value, correct the first data stored in the memory based on the first deviation information, obtain the biometric information measurement value corresponding to the first color value based on the corrected first data, and obtain a diagnostic result corresponding to the obtained biometric information measurement value based on the second data stored in the memory.

In accordance with another aspect of the disclosure, a control method of an electronic device is provided. The control method includes obtaining, based on a user command for photographing a diagnostic kit including at least one test line discolored according to a reaction between a sample collected from a body of a user and a reagent and a plurality of tone distinction markers with different tones from one another being received, an image on the diagnostic kit through a camera included in the electronic device, obtaining a first color value on the at least one test line included in the obtained image and a second color value on each of the plurality of tone distinction markers included in the obtained image, obtaining a first deviation information on a photographing environment of the obtained image based on the second color value, correcting a first data to obtain a biometric information measurement value according to the first color value stored in the electronic device based on the first deviation information, obtaining the biometric information measurement value corresponding to the first color value based on the corrected first data, and obtaining a diagnostic result corresponding to the obtained biometric information measurement value based on a second data for obtaining the diagnostic result according to the biometric information measurement value stored in the electronic device.

In accordance with another aspect of the disclosure, a non-transitory computer readable recording medium is provided. The medium includes a program executing a control method of an electronic device, the control method of the electronic device includes obtaining, based on a user command for photographing a diagnostic kit including at least one test line discolored according to a reaction between a sample collected from a body of a user and a reagent and a plurality of tone distinction markers with different tones from one another being received, an image on the diagnostic kit through a camera included in the electronic device, obtaining a first color value on the at least one test line included in the obtained image and a second color value on each of the plurality of tone distinction markers included in the obtained image, obtaining a first deviation information on a photographing environment of the obtained image based on the second color value, correcting a first data to obtain a biometric information measurement value according to the first color value stored in the electronic device based on the first deviation information, obtaining the biometric information measurement value corresponding to the first color value based on the corrected first data, and obtaining a diagnostic result corresponding to the obtained biometric information measurement value based on a second data for obtaining the diagnostic result according to the biometric information measurement value stored in the electronic device.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a concept view illustrating in brief an electronic device and a diagnostic kit according to an embodiment of the disclosure;

FIG. 2 is a view illustrating a test line and a plurality of tone distinction markers according to an embodiment of the disclosure;

FIG. 3 is a flowchart illustrating a control method of an electronic device according to an embodiment of the disclosure;

FIG. 4A is a view illustrating an identification marker on a manufacturing deviation of a diagnostic kit according to an embodiment of the disclosure;

FIG. 4B is a view illustrating an identification marker on a manufacturing deviation of a diagnostic kit according to an embodiment of the disclosure;

FIG. 5 is a view illustrating an embodiment related to a case in which a diagnostic kit includes an identification marker on a manufacturing deviation of a diagnostic kit according to an embodiment of the disclosure;

FIG. 6 is a view illustrating in detail a process of correcting first data based on first deviation information and second deviation information according to an embodiment of the disclosure;

FIG. 7 is a view illustrating an embodiment related to a personalization of second data according to an embodiment of the disclosure;

FIG. 8 is a view illustrating an embodiment related to obtaining a diagnostic result based on a plurality of biometric information measurement values different from one another according to an embodiment of the disclosure;

FIG. 9 is a view illustrating an embodiment related to a method of photographing a diagnostic kit by using an electronic device according to an embodiment of the disclosure;

FIG. 10 is a block diagram illustrating in brief a configuration of an electronic device according to an embodiment of the disclosure; and

FIG. 11 is a block diagram illustrating in detail a configuration of an electronic device according to an embodiment of the disclosure.

The same reference numerals are used to represent the same elements throughout the drawings.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

In case it is determined that in describing embodiments, detailed description of related known technologies may unnecessarily confuse the gist of the disclosure, the detailed description may be omitted.

In addition thereto, the embodiments below may be modified to various forms, and the technical scope of disclosure is not limited to the embodiments described below. Rather, the embodiments more faithfully complete the disclosure, and are provided to fully convey the technical idea of the disclosure to a person of ordinary skill in the art.

The terms used herein are used merely to describe a specific embodiment, and is not intended to limit the scope of protection. A singular expression may include a plural expression, unless otherwise specified.

Expressions such as, for example, and without limitation, “comprise,” “may comprises,” “include,” “may include,” or the like used herein may designate a presence of a corresponding characteristic (e.g., elements such as a number, a function, an operation or a component), and not preclude a presence of additional characteristics.

In the disclosure, expressions such as “A or B,” “at least one of A and/or B,” or “one or more of A and/or B” may include all possible combinations of the items listed together. For example, “A or B,” “at least one of A and B,” or “at least one of A or B” may refer to all cases including (1) at least one of A, (2) at least one of B, or (3) both of at least one of A and at least one of B.

Expressions such as “first,” “second,” “1st,” “2nd,” or so on may be used on various elements regardless of order and/or importance, and used only to distinguish one element from another, and not limit the corresponding elements.

When a certain element (e.g., first element) is indicated as being “(operatively or communicatively) coupled with/to” or “connected to” another element (e.g., second element), it may be understood as the certain element being directly coupled with/to the other element or as being coupled through still another element (e.g., third element).

On the other hand, when a certain element (e.g., first element) is indicated as “directly coupled with/to” or “directly connected to” another element (e.g., second element), it may be understood as still another element (e.g., third element) not being present between the certain element and the other element.

The expression “configured to . . . (or set up to)” used in the disclosure may be used interchangeably with, for example, “suitable for . . . ,” “having the capacity to . . . ,” “designed to . . . ,” “adapted to . . . ,” “made to . . . ,” or “capable of . . . ” based on circumstance. The term “configured to . . . (or set up to)” may not necessarily mean “specifically designed to” in terms of hardware.

Rather, in a certain circumstance, the expression “a device configured to . . . ” may mean something that the device “may perform . . . ” together with another device or components. For example, the phrase “a processor configured to (or set up to) perform A, B, and C” may mean a dedicated processor (e.g., embedded processor) for performing a corresponding operation, or a generic-purpose processor (e.g., a central processing unit (CPU) or an application processor) capable of performing the corresponding operations by executing one or more software programs stored in a memory device.

In the embodiments, a ‘module,’ or a ‘part’ may perform at least one function or an operation, and may be implemented as hardware or software, or a combination of hardware and software. In addition, a plurality of ‘modules’ or a plurality of ‘parts’ may be integrated in at least one module and realized in at least one processor except for when a ‘module’ or a ‘part’ needs to be realized in an individual hardware.

Various elements and areas in the drawings have been schematically illustrated. Accordingly, the technical idea of the disclosure is not limited by the relative size or distance illustrated in the accompanied drawings.

The electronic device according to the various embodiments may include at least one from among a smartphone, a tablet personal computer (PC), a desktop computer, a laptop PC, or a wearable device. The wearable device may include at least one of an accessory type (e.g., a watch, a ring, a bracelet, an anklet, a necklace, a pair of glasses, a contact lens or a head-mounted-device (HMD)), a fabric or a garment-embedded type (e.g., an electronic clothing), a body-attached type (e.g., a skin pad or a tattoo), or a bio-implantable circuit.

In some embodiments, the electronic device may include at least one from among, for example, and without limitation, a television, a digital video disk (DVD) player, an audio, a refrigerator, a cleaner, an oven, a microwave, a washing machine, an air purifier, a set top box, a home automation control panel, a security control panel, a media box (e.g., SAMSUNG HOMESYNC™, APPLE TV™, or GOOGLE TV™), a game console (e.g., XBOX™, PLAYSTATION™, etc.), an electronic dictionary, an electronic key, a camcorder, or an electronic frame.

In another embodiment, the electronic device may include at least one from among various medical devices (e.g., various portable medical measurement device (e.g., glucose measuring device, a heart rate measuring device, a blood pressure measuring device, a temperature measuring device, etc.), a magnetic resonance angiography (MRA), a magnetic resonance imaging (MRI), a computed tomography (CT), an imaging apparatus, an ultrasonic device, etc.), a navigation device, a global navigation satellite system (GNSS), an event data recorder (EDR), a flight data recorder (FDR), a vehicle infotainment device, a nautical electronic equipment (e.g., nautical navigation device, gyro compass, etc.), an avionics electronic device, a security device, a vehicle head unit, an industrial or personal robot, a drone, an automated teller machine (ATM) of financial institutions, a point of sales (POS) of shops, or an internet of things device (e.g., light bulbs, various sensors, sprinkler devices, fire alarms, temperature adjusters, street lights, toasters, exercise equipment, hot water tanks, heater, boilers, etc.).

The embodiments will be described in greater detail below with reference to the accompanying drawings to assist those of ordinary skill in the art to easily understand the disclosure.

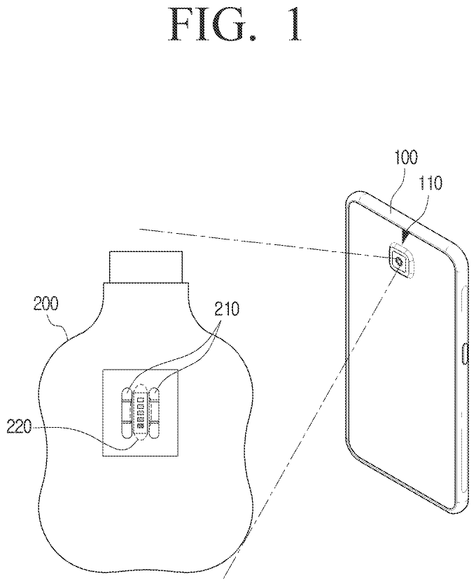

FIG. 1 is a concept view illustrating in brief an electronic device and a diagnostic kit according to an embodiment of the disclosure.

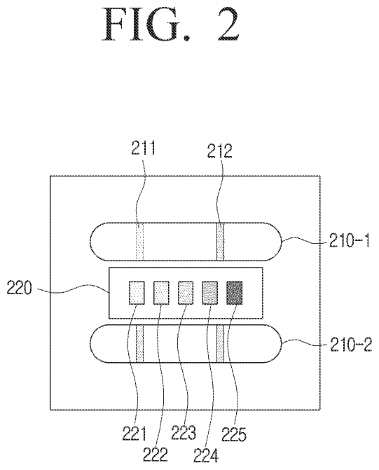

FIG. 2 is a view illustrating a test line and a plurality of tone distinction markers according to an embodiment of the disclosure.

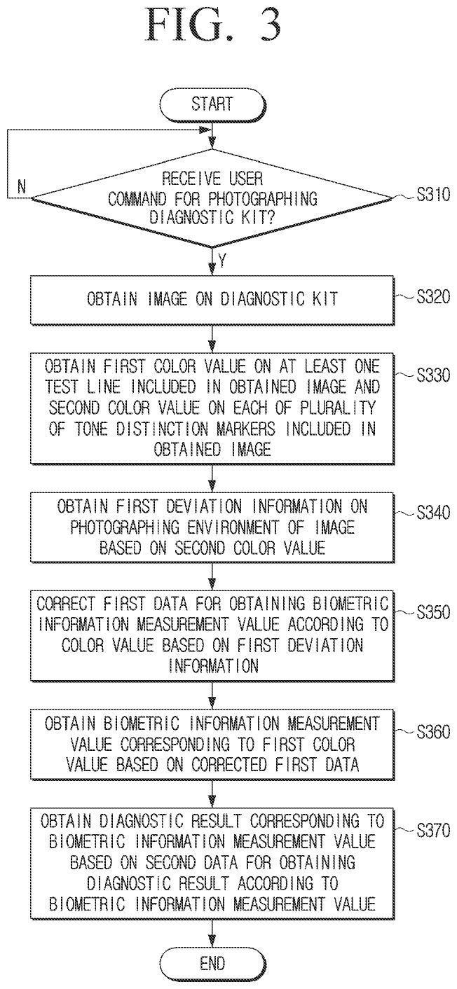

FIG. 3 is a flowchart illustrating a control method of an electronic device according to an embodiment of the disclosure.

Referring to FIG. 1, first, the ‘electronic device 100’ according to an embodiment of the disclosure may refer to a device capable of obtaining an image on a diagnostic kit 200 by photographing the diagnostic kit 200, and obtaining a diagnostic result by analyzing the obtained image. For example, the electronic device 100 may be implemented as a smart phone as illustrated in FIG. 1. However, the electronic device 100 according to the disclosure is not limited to the electronic device 100 of a specific type, and so long as it is a device capable of obtaining an image on the diagnostic kit 200 by photographing the diagnostic kit 200 and obtaining a diagnostic result by analyzing the obtained image, it may all correspond to the electronic device 100 according to the disclosure.

The ‘diagnostic kit 200’ according to the disclosure may refer to a diagnostic equipment capable of showing a reaction result between a sample collected from a body of a user and a reagent provided in the diagnostic kit 200. Specifically, when a sample such as saliva, blood, urine or exhaled breath collected from the body of the user is added to the diagnostic kit 200, the test line 211 included in the diagnostic kit 200 may be discolored according to a reaction between the sample and the reagent. Then, the electronic device 100 may analyze the color of the discolored test line 211 of the diagnostic kit 200 quantitively and provide the diagnostic result.

In order to provide the diagnostic result by analyzing the color of the discolored test line 211, a first data for obtaining a biometric information measurement value according to a color value and a second data for obtaining the diagnostic result according to the biometric information measurement value may be stored in the electronic device 100 according to the disclosure.

The ‘first data’ may include a function capable of outputting the biometric information measurement value corresponding to the input color value or information on a lookup table, and the ‘second data’ may include information on a function or a lookup table capable of outputting the diagnostic result according to a section in which the input the biometric information measurement value is included from among a plurality of pre-defined sections. Specifically, the first data and the second data may be received from an external device together with an application which is implemented so that the control method according to the disclosure may be performed and stored in the electronic device 100.

The ‘biometric information measurement value’ may refer to a value obtained by quantitively analyzing the biometric information which is the subject of measurement, and for example, may refer to a concentration value of a hormone which is the subject of measurement.

The ‘color value,’ which converts and quantifies a color characteristic to a coordinate value of a geometric color space, may include, for example, a red, green and blue (RGB) value, a cyan magenta yellow and black (CMYK) value, or the like. The ‘biometric information measurement value’ may be a concentration value of a biological hormone, and the ‘diagnostic result’ may be information indicating whether it is negative or positive with respect to a disease subject to diagnosis. However, the color information, the biometric information measurement value and the diagnostic result according to the disclosure may not be limited to the above-described examples.

Each operation of the control method for providing an accurate diagnostic result by analyzing the color of the test line 211 quantitatively will be described below with reference to FIGS. 2 to 3.

Referring to FIG. 3, the electronic device 100 may receive a user command for photographing the diagnostic kit 200 at operation S310. Then, based on receiving the user command for photographing the diagnostic kit 200, the electronic device may obtain an image of the diagnostic kit 200 through the camera 110 at operation S320.

The diagnostic kit 200 may include at least one reagent pad 210 and a plurality of tone distinction markers 220 as illustrated in FIG. 1. The ‘reagent pad 210’ may be disposed inside the diagnostic kit 200 in a strip form including a reagent, but as illustrated in FIGS. 1 and 2, a part of the reagent pad 210 may be exposed outside of the diagnostic kit 200. The reagent pad 210 may be substituted with terms such as bio sensor.

Referring to FIG. 2, at least one reagent pad 210-1 and 210-2 may include each of a test line 211 and a control line 212. The ‘test line 211’ may refer to a line which appears as the reagent pad 210 is discolored according to a reaction between a sample collected from a body of a user and a reagent, and represent an amount of biometric information which is the subject of measurement according to a degree of discoloration. For example, the test line 211 may be discolored to a red color according to a chemical reaction being carried out between the sample collected from the user body and the reagent provided in the diagnostic kit 200. Then, the tone of the color that appeared on the test line 211, that is a brightness or saturation, may be varied according to the concentration of biometric information, which is the subject of measurement.

The ‘control line 212’ may be a line which represents whether the corresponding reagent pads 210-1 and 210-2 are in a normal state, and the control line 212 may be discolored to a predetermined color if the reaction between the sample and the reagent occurs normally. On the other hand, if the control line 212 is not discolored even after adding the sample in the diagnostic kit 200, it may be verified that the corresponding reagent pad 210 is not in a normal state.

The ‘plurality of tone distinction markers 220’ may, as a reference marker for comparison with the test line 211, have a same color as the color of the discolored test line 211, and may be configured to have a brightness saturation different from each other. As illustrated in FIG. 2, the plurality of tone distinction markers 220 may be comprised of five tone distinction markers 221, 222, 223, 224 and 225, and each of the five tone distinction markers 221, 222, 223, 224 and 225 may all be configured to have a red color which is the same color as the discolored test line 211, yet have a discrete 5 stage brightness or saturation.

Then, the plurality of tone distinction markers 220 may be included within one image together with the test line 211, and further, it may be preferable for the plurality of tone distinction markers 220 to be disposed at a location adjacent to the test line 211 on the diagnostic kit 200 so that it may be photographed under lighting environment similar to the lighting environment of the test line 211. As illustrated in FIG. 2, if the diagnostic kit 200 includes two reagent pads 210, it may be preferable for the plurality of tone supplementary markers to be disposed between the two reagent pads 210 so that the plurality of tone supplementary markers may be included in the image on the diagnostic kit together with the two reagent pads 210.

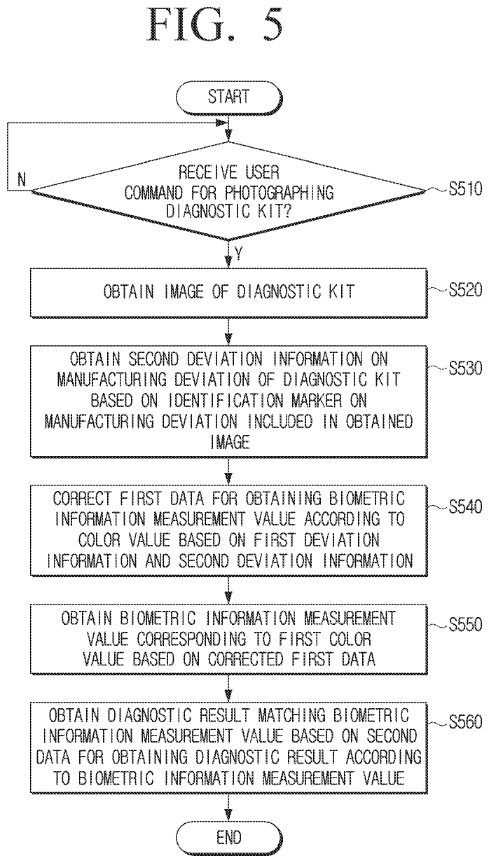

In the description of FIGS. 1 to 3, only the embodiment of the diagnostic kit 200 including the plurality of tone distinction markers 220 will be described, but the diagnostic kit 200 according to the disclosure may include an identification marker on a manufacturing deviation. The embodiment related to the diagnostic kit 200 including the identification marker on the manufacturing deviation will be described with reference to FIG. 5.

When an image on the diagnostic kit 200 is obtained, the electronic device 100 may obtain a first color value on at least one test line 211 included in the obtained image and a second color value on each of the plurality of tone distinction markers 220 included in the obtained image at operation S330. Specifically, the electronic device 100 may identify the at least one test line 211 included in the obtained image, and obtain an RGB value on the identified at least one test line 211, respectively.

If the first data includes a function capable of outputting the biometric information measurement value as described above, the electronic device 100 may obtain the biometric information measurement value corresponding to the first color value by inputting the obtained first color value to the function thereof. However, based on the environment at the time of photographing the image on the diagnostic kit 200, because at least one from among the brightness and saturation of the test line 211 included in the image may appear different from the at least one from among the brightness and saturation of the test line 211 appearing in the actual diagnostic kit 200, the effect according to the environment at the time of photographing with respect to obtaining the biometric information measurement value corresponding to the obtained first color value needs to be considered.

Accordingly, the diagnostic kit 200 according to the disclosure may include a plurality of tone distinction markers 220 as described above, and the electronic device 100 may identify the plurality of tone distinction markers 220 included in the obtained image, and obtain the second color value on each of the identified plurality of tone distinction markers 220 and use it to correct the first data. Specifically, the electronic device 100 may obtain a different second color value on each of the plurality of tone distinction markers based on the different brightness and saturation of each of the plurality of tone distinction markers included in the image on the diagnostic kit. Then, the electronic device 100 may obtain, based on the second color value, a first deviation information on a photographing environment of the obtained image at operation S340, and correct, based on the first deviation information, the first data stored in the memory at operation S350.

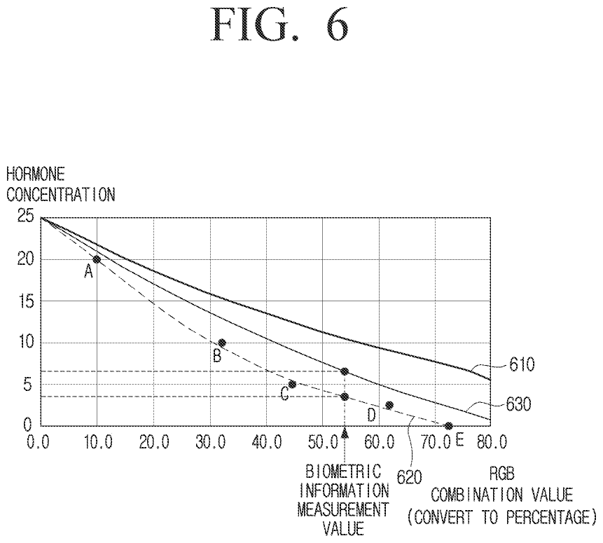

The ‘first deviation information’ may be a term for collectively designating information on environmental elements which may influence the test line 211 included in the image and the color value of a tone supplementary marker. For example, the first deviation information may include information on the color of the surrounding light sources at the time of photographing the image, information on the amount of light of the surrounding light sources, and the like. The correcting first data based on first deviation information may refer to, for example, determining a final function for using the biometric information measurement value by changing a coefficient of the function included in the first data with a value corresponding to the obtained first deviation information. The process of correcting the first data based on the first deviation information will be described in greater detail below with reference to FIG. 6.

When the first data is corrected, the electronic device 100 may obtain, based on the corrected first data, the biometric information measurement value corresponding to the obtained first color value at operation S360. For example, when the final function is determined by correcting the first data based on the second color value as described above, the electronic device 100 may input the obtained first color value to the determined function and obtain the biometric information measurement value corresponding to the first color value. For example, the electronic device 100 may input a percentage value of 48.99 of an R value with respect to (170, 84, 93) which is the obtained first color value (RGB value), and obtain information that a concentration of a hormone, which is the subject of measurement, is 5 ng/mL.

When the biometric information measurement value corresponding to the first color value is obtained, the electronic device 100 may obtain a diagnostic result corresponding to the obtained biometric information measurement value based on the second data at operation S370. The electronic device 100 may obtain, based on which section from among a plurality of pre-defined numerical sections in the second data the biometric information measurement value belongs, the diagnostic result corresponding to the input biometric information measurement value. For example, based on obtaining information that the concentration of the hormone, which is the subject of measurement as described in the example above, is 5 ng/mL, the electronic device 100 may identify that the obtained biometric information measurement value of 5 ng/mL falls within 3 ng/mL to 9 ng/mL, which is the section pre-defined as corresponding to a diagnostic result of ‘normal’, and obtain the diagnostic result of ‘normal.’

When the diagnostic result is obtained as described above, the electronic device 100 may provide the obtained diagnostic result to the user. Specifically, the electronic device 100 may visually display the obtained diagnostic result through a display provided in the electronic device 100, and output in speech form through a speaker included in the electronic device 100. In addition thereto, the electronic device 100 may provide the biometric information measurement value itself to the user.

According to the embodiment as described above with reference to FIGS. 1 to 3, the electronic device 100 may more accurately analyze the quantitative value on the color of the test line 211 apparent in the diagnostic kit 200 and provide the diagnostic result by obtaining information on the photographing environment of the image based on the color value on the tone supplementary marker disposed in the diagnostic kit 200.

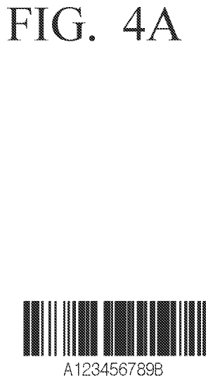

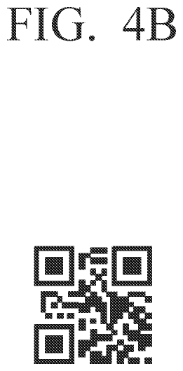

FIGS. 4A and 4B are views illustrating an identification marker on a manufacturing deviation of a diagnostic kit according to various embodiments of the disclosure.

FIG. 5 is a view illustrating an embodiment related to a case in which a diagnostic kit includes an identification marker on a manufacturing deviation of a diagnostic kit according to an embodiment of the disclosure.

Referring to FIGS. 1 to 3, the diagnostic kit 200 according to the disclosure may include a plurality of tone supplement markers, and an embodiment related to the electronic device 100 using the first deviation information included in the plurality of tone markers has been described, but the diagnostic kit 200 according to the disclosure may include an identification marker on the manufacturing deviation and thus, the image on the diagnostic kit 200 may also include the identification marker on the manufacturing deviation.

The ‘identification marker on the manufacturing deviation’ may, as a marker for reflecting the influence according to the manufacturing deviation which may be generated in the manufacturing process of the diagnostic kit 200 in obtaining the biometric information measurement value corresponding to the first color value, be disposed on the diagnostic kit 200 in the form of a barcode as illustrated in FIG. 4A or a quick response code (QR code) as illustrated in FIG. 4B. Specifically, it may be preferable for the identification marker on the manufacturing deviation to be disposed at location adjacent to the test line on the diagnostic kit 200 so that it may be included within one image together with the test line and the plurality of tone distinction markers.

Referring to FIG. 5, even if the diagnostic kit 200 includes the identification marker on the manufacturing deviation, the electronic device 100 may obtain, based on receiving a user command for photographing the diagnostic kit 200 at operation S510, an image on the diagnostic kit 200 through the camera at operation S520. Then, when the image on the diagnostic kit 200 is obtained at operation S510—Y, the electronic device 100 may obtain second deviation information on the manufacturing deviation of the diagnostic kit 200 based on the identification marker on the manufacturing deviation included in the obtained image at operation S530, and correct first data based on the second deviation information at operation S540.

The ‘second deviation information’ may be a term for collectively designating information on the manufacturing deviation which is apparent in a final product according to an error generated by a chemical/physical error which may be generated in the manufacturing process of the diagnostic kit 200. Specifically, the second deviation information may be seen as a type of tolerance, and may be obtained by testing per batch on whether a degree of deviation has occurred compared to a required specification or a standard specification through a final quality control operation from among the manufacturing process of the diagnostic kit 200.

More specifically, in a final operation of the manufacturing process of the diagnostic kit 200, when information on a calibration curve per lot, which is a manufacturing unit having the same characteristic, is obtained, the information on the obtained calibration curve may be inserted in the identification marker on the manufacturing deviation in the form of a barcode or a QR code. Then, the electronic device may obtain information on the calibration curve of the diagnostic kit based on the barcode or QR code included in the image on the diagnostic kit, and obtain the second deviation information based on information on the obtained calibration curve.

The correcting first data based on the second deviation information may refer to, for example, determining the final function for using in the biometric information measurement value by changing the coefficient of the function included in the data as a value corresponding to the obtained second deviation information. The process of correcting the first data based on the second deviation information will be described in greater detail with reference to FIG. 6.

When the first data is corrected based on the second deviation information, the electronic device 100 may obtain a biometric information measurement value corresponding to the obtained first color value based on the corrected first data, as in the embodiment in which the diagnostic kit 200 includes a plurality of tone distinction markers at operation S550, and obtain a diagnostic result corresponding to the obtained biometric information measurement value based on the second data at operation S560.

In the above, an embodiment of the electronic device 100 correcting first data based on first deviation information has been described with reference to FIGS. 1 to 3, and an embodiment of the electronic device 100 correcting second data based on second deviation information have been described with reference to FIGS. 4A to 5, but the disclosure is not limited to the embodiments described. That is, because the electronic device 100 may correct first data considering both the first deviation information and the second deviation information, the embodiment will be described in greater detail with reference to FIG. 6.

According to an embodiment as described above with reference to FIGS. 4A to 5, the electronic device 100 according to the disclosure may more accurately analyze the quantitative value on the color of the test line apparent in the diagnostic kit 200 taking into consideration information on the manufacturing deviation of the diagnostic kit 200 and provide a diagnostic result corresponding thereto.

FIG. 6 is a view illustrating in detail a process of correcting first data based on first deviation information and second deviation information according to an embodiment of the disclosure.

Referring to FIG. 6, the first graph 610 illustrated in FIG. 6 may represent, based on the function included in the first data being pre-defined as a form of a cubic function, a graph on an optional function f(x)=a₁x³+b₁x²+c₁x+d₁ corresponding to first data prior to correction. The x-axis in FIG. 6 may represent a percentage value of the R value in the RGB value and the y-axis may refer to the biometric information measurement value corresponding to x.

According to the related art, the electronic device 100 may output the biometric information measurement value corresponding to the color value based on the function pre-stored in the electronic device 100 as with the first graph 610 without having to consider the first deviation information on the photographing environment of the image or the second deviation information on the manufacturing deviation of the diagnostic kit 200.

The second graph 620 illustrated in FIG. 6 may represent a graph corresponding to the first data corrected based on the first deviation information. Specifically, the electronic device 100 may obtain the second color value on each of the plurality of tone distinction markers included in the image on the diagnostic kit 200, and obtain the first deviation information on the photographing environment of the image based on the obtained second color value. For example, the electronic device 100 may obtain the first deviation information including information that the color temperature of the surrounding light source may be 5900 K and information that the light amount of the surrounding light source may be 300 lx.

The first data may include information on a function capable of outputting the biometric information measurement value corresponding to the input color value, and the information on the type of function used in the obtaining of the biometric information measurement value and the information on the coefficient of the function according to the first deviation information may be pre-defined according to the biometric information, which is the subject of measurement. For example, the electronic device 100 may be stored with information that the function used in obtaining the biometric information measurement value of hormone A is cubic function.

The electronic device 100 may be stored with information on the coefficient of the function corresponding to the obtained first deviation information, and the electronic device 100 may determine the function used in the biometric information measurement value based on information on the coefficient of the function corresponding to the first deviation information. For example, if the first deviation information including information that the color temperature of the surrounding light source is 5900 K and information that the light amount of the surrounding light source is 300 lx is obtained, the electronic device 100 may determine the function used in the biometric information measurement value based on the value of coefficient a₂, b₂, c₂ and d₂ of the function corresponding thereto.

In addition thereto, the electronic device 100 may be stored with the biometric information measurement value corresponding to each of the second color value on the plurality of tone distinction markers, and in this case, the electronic device 100 may determine the function used in the biometric information measurement value based on the biometric information measurement value corresponding to each of the second color value on the plurality of tone distinction markers. For example, a coordinate representing the second color value on each of the plurality of tone distinction markers and the biometric information measurement value corresponding thereto may be equivalent to A, B, C, D and E as illustrated in FIG. 6. The electronic device 100 may determine the value of coefficients a₂, b₂, c₂ and d₂ of the function so that the function includes coordinates A, B, C, D and E.

When correcting the first data by using only the first deviation information as described above, the function corresponding to the second graph 620 as illustrated in FIG. 6 may be determined as the function finally used in the biometric information measurement value. However, the electronic device 100 according to the disclosure may use the second deviation information together with the first deviation information to correct the first data, and in this case, the function corresponding to the third graph 630 as illustrated in FIG. 6 may be determined as the function finally used in biometric information measurement value.

That is, the third graph 630 illustrated in FIG. 6 may represent a graph corresponding to the first data corrected based on the first deviation information and the second deviation information. As described above, the electronic device 100 may recognize the barcode or the QR code which is the identification marker included in the image on the diagnostic kit 200 and obtain the second deviation information on the manufacturing deviation of the diagnostic kit 200.

The electronic device 100 may be stored with the first deviation information and information on the coefficient of the function corresponding to the second deviation information. That is, the electronic device 100 may not only be stored with information on the coefficient of the function corresponding to each of the photographing environments apparent by the first deviation information, but may also be pre-stored with information on the coefficient of the which function is to be determined according to the manufacturing deviation of the diagnostic kit 200 different from one another based on the premise of a specific photographing environment. Accordingly, the electronic device 100 may finally determine the function used in the biometric information measurement value based on information on the coefficient of the function corresponding to the first deviation information and the second deviation information. In other words, the electronic device 100 according to the disclosure may accurately analyze the quantitative value on the color of the test line apparent in the diagnostic kit 200 taking into consideration all of the photographing environment of the image and the information on the manufacturing deviation of the diagnostic kit 200.

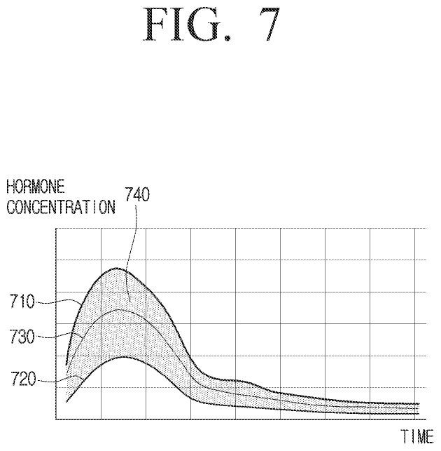

FIG. 7 is a view illustrating an embodiment related to a personalization of second data according to an embodiment of the disclosure.

Although the embodiment of obtaining the diagnostic result based on the pre-stored second data has been described with respect to the description of FIGS. 1 to 6, the second data according to the disclosure may be personalized by being updated each time the biometric information measurement value on the user and the diagnostic result are obtained. The ‘personalization’ of second data may refer to the range of second data used in finally obtaining the diagnostic result being extended and determined to be appropriate for the individual user as the biometric information measurement value per user and the diagnostic result are accumulatively stored.

Each operation of the control method as described above may be repeated multiple times with respect to a specific user, and the electronic device 100 may store the biometric information measurement value on the specific user each time it is obtained. Then, based on a plurality of biometric information measurement values on the specific user being stored and then new biometric information value on the user being obtained, the electronic device 100 may compare the pre-stored plurality of biometric information measurement values with the new biometric information measurement value, and obtain the diagnostic result according to the comparison result.

Referring to FIG. 7, a graph representing the concentration of a stress hormone (e.g., cortisol) of a specific user according to the passing of time during one day is illustrated, and specifically, the first graph 710 may represent the hormone concentration obtained during the first day of testing, and the second graph 720 may represent the hormone concentration obtained during the second day of testing. Then, the third graph 730 may represent the average value of the hormone concentration obtained during the first day of testing and the hormone concentration obtained during the second day of testing.

Based on the hormone concentration value being obtained at the third day of testing from the same user after the hormone concentration value as illustrated in FIG. 7 is obtained, the electronic device 100 may compare the hormone concentration value corresponding to the first graph 710, the second graph 720, and the third graph 730 with the hormone concentration value obtained at the third day of testing, and provide the diagnostic result including whether the stress of the user has improved or worsened compared to the first day of testing, the second day of testing or the average of the first day of testing and the second day of testing. In addition, the electronic device may provide a diagnostic result related to whether the hormone concentration value obtained at the third day of testing is within a range 740 of the hormone concentration obtained at the first day of testing and the second day of testing.

According to an embodiment as described above with reference to FIG. 7, the electronic device 100 is not configured to provide the diagnostic result based on a theoretical/statistical determination criterion, but may provide a more accurate diagnostic result by reflecting the intrinsic characteristic of the individual user with respect to the biometric information based on the personalized data for each user.

Further, if information on activities such as exercise, sleep and therapy performed by the user from the first day of testing to the third day of testing is additionally input, the electronic device 100 may provide a result related to what influence the activities performed by the user has had on the change in stress along with the diagnostic result.

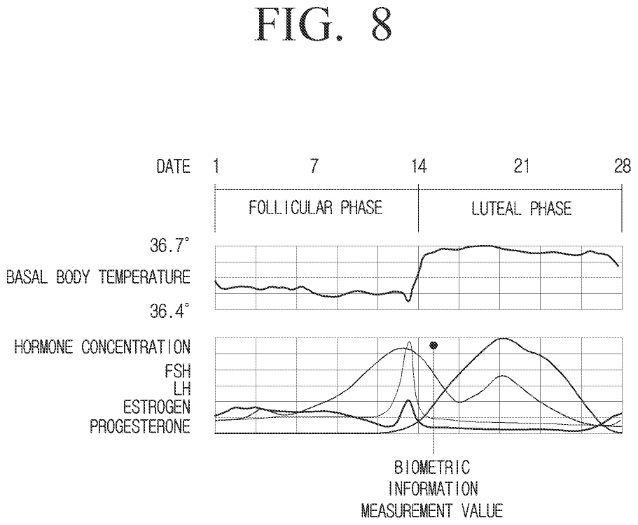

FIG. 8 is a view illustrating an embodiment related to obtaining a diagnostic result based on a plurality of biometric information measurement values different from one another according to an embodiment of the disclosure.

Although the embodiment related to providing a diagnostic result based on the measurement value on one specific biometric information has been described in describing FIGS. 1 to 7, a measurement value on the plurality of biometric information may be needed to provide an accurate diagnostic result on problems determined according to a complex interaction of hormones of various types such as an ovulation date or a menstruation date of women in particular.

Accordingly, the diagnostic kit 200 according to the disclosure may include a plurality of reagent pads as illustrated in FIG. 2, and the each of the plurality of reagent pads may be configured to measure a plurality of biometric information different from one another. The electronic device 100 may analyze the test line included in each of the plurality of reagent pads and obtain the plurality of biometric information measurement values. In addition thereto, even if the diagnostic kit 200 is configured to measure one biometric information, the electronic device 100 may analyze the image on the plurality of diagnostic kits 200 configured to measure the each of the different biometric information from one another and obtain the plurality of biometric information measurement values.

Referring to FIG. 8, a graph representing the plurality of hormone concentrations with respect to a specific user according to the passing of time for one month is illustrated, and specifically, may represent the concentration values of each of a follicle stimulating hormone (FSH), a luteinizing hormone (LH), an estrogen, and a progesterone which are the plurality of hormones capable of influencing the menstruation cycle of women. Specifically, the graphs of FIG. 8 may not only be graphs obtained based on the statistical data, but as described with reference to FIG. 7, may also be graphs corresponding to personalized data based on the hormone concentration values of a specific user.

When the concentration value on the FSH, the LH, the estrogen, and the progesterone corresponding to a vertical line marked as the ‘biometric information measurement value’ in FIG. 8 is newly obtained from the user while the data corresponding to the graph of FIG. 8 is in a stored state, the electronic device 100 may compare the pre-stored concentration values with the new concentration values and provide a diagnostic result according to the comparison result.

Specifically, the electronic device 100 may obtain information that the newly obtained concentration value is obtained at the 15^{th} day of the menstruation cycle, information that ovulation was induced as the LH increased in the body of the user, information that the concentration of progesterone has begun to increase as ovulation occurred, and the like. Accordingly, the electronic device 100 may finally provide a diagnostic result that the user is in a fertile phase. Additionally, the electronic device 100 may, in providing the diagnostic result that the user is in a fertile phase, use information that a basal body temperature of the user has increased as illustrated in FIG. 8.

As described above, because the ovulation date or the menstruation date of women may be determined differently according to a hormonal characteristic of each user or deviation, when information on the concentration of each of the FSH, LH, estrogen and progesterone of a specific user is stored as described with reference to FIG. 7, the electronic device 100 may provide a more accurate diagnostic result based on the personalized data with respect to the specific user.

According to an embodiment as described above with reference to FIG. 8, unlike broadly specifying the fertility period (3-5 days) based on a statistical determination criterion (25 mIU/ml) as in the case of related art, a more accurate diagnostic result may be provided based on information on a plurality of hormones which may influence the ovulation date of women.

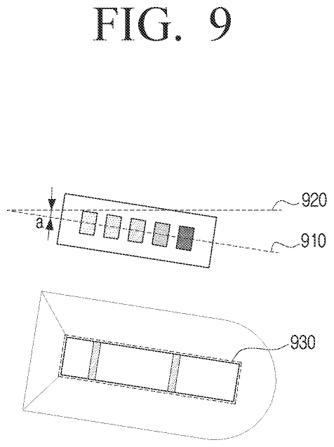

FIG. 9 is a view illustrating an embodiment related to a method of photographing a diagnostic kit by using an electronic device according to an embodiment of the disclosure.

In obtaining the color value on the test line by analyzing the image on the diagnostic kit 200 as in the disclosure, an interest area including the test line in the image may be identified, and it may be preferable for the edges of the interest area to be parallel with the edges of the image. This is because, in applying the image processing technology for obtaining the color value on the test line, the image which is the subject of analysis is processed by dividing into a plurality of square shape areas according to the plurality of vertical lines and horizontal lines.

According to the related art, the electronic device 100 may display a UI element corresponding to the interest area on a user interface (UI) of an application for photographing the diagnostic kit 200, and has been implemented so that a normal image photographing or measuring of a normal biometric information measurement value is possible so long as the UI element and the reagent pad part of the diagnostic kit 200 matches within a pre-set threshold range. However, according to an embodiment of the disclosure, normal image photographing or measuring of a normal biometric information measurement value may be possible without the provision of the UI element according to the related art.

Referring to FIG. 9, when the image on the diagnostic kit 200 is obtained, the electronic device 100 may identify a line 910 which connects a center point of each of the plurality of tone supplementary markers included in the image and an angle a between the horizontal lines 920 which divide the image. If the edges of the plurality of tone supplementary markers and the edges of the reagent pad within the diagnostic kit 200 are parallel with one another, the identified angle a may be the same as the angle at which the reagent pad is inclined within the diagnostic kit 200. Accordingly, the electronic device 100 may identify the interest area 930 based on the identified angle a and the distance between the plurality of tone supplementary markers and the reagent pad.

According to an embodiment of the disclosure as described above with reference to FIG. 9, user convenience may be improved by not having to photograph the reagent pad included in the diagnostic kit 200 arranged to a specific direction in the process of photographing the image on the diagnostic kit 200.

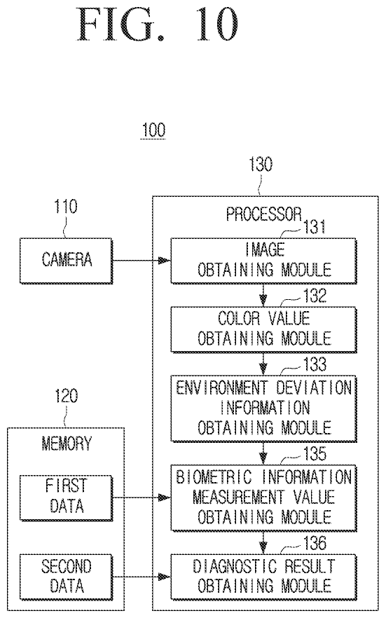

FIG. 10 is a block diagram illustrating in brief a configuration of an electronic device according to an embodiment of the disclosure.

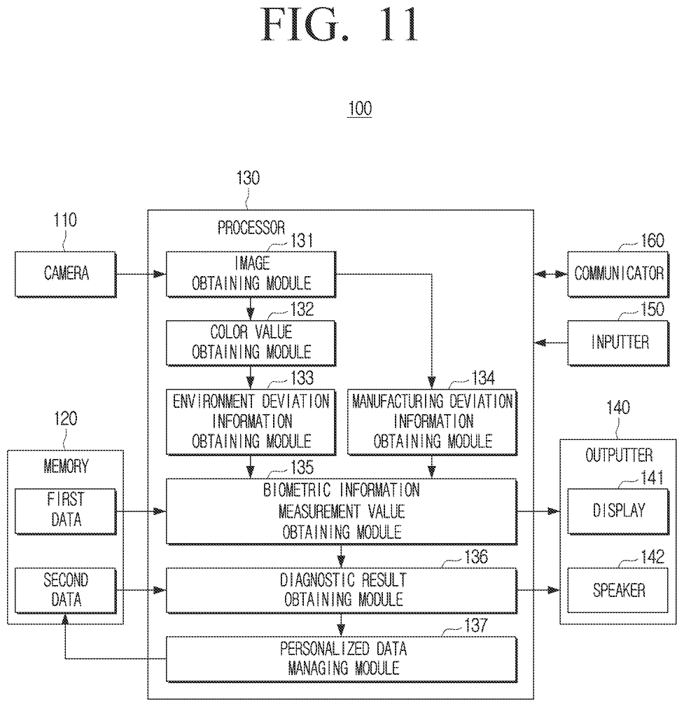

FIG. 11 is a block diagram illustrating in detail a configuration of an electronic device according to an embodiment of the disclosure.

Referring to FIG. 10, the electronic device 100 according to the disclosure may include a hardware configuration such as a camera 110, a memory 120, and a processor 130. Referring to FIG. 11, the electronic device 100 may further include an outputter 140, an inputter 150, and a communicator 160.

The processor 130 may include modules such as an image obtaining module 131, a color value obtaining module 132, an environment deviation information obtaining module 133, a manufacturing deviation information obtaining module 134, a biometric information measurement value obtaining module 135, a diagnostic result obtaining module 136, and a personalized data managing module 137 as illustrated in FIGS. 10 and 11. These modules may be stored in the memory 120 as software modules configured to implement various operations according to the disclosure, and may be used after being loaded to the processor 130 by the control of the processor 130. However, at least some from among a plurality of modules illustrated in FIGS. 10 and 11 may be implemented as a hardware module.

The hardware configuration and software module as illustrated in FIGS. 10 and 11 may be mutually coupled organically and configured so as to implement the various embodiments according to the disclosure. The configurations as illustrated in FIGS. 10 and 11 are merely examples, and in implementing the disclosure, a new configuration may be added to or a part of the configuration may be omitted from the configurations as illustrated in FIGS. 10 and 11. Each configuration and module of FIGS. 10 and 11 will be described below.

The camera 110 may obtain an image including at least one object. Specifically, the camera 110 may include an image sensor, and the image sensor may convert light introduced through the lens to an electric image signal.

According to the various embodiments of the disclosure, the processor 130 may be configured to obtain the image on the diagnostic kit 200 through the camera 110, and accordingly obtain an image on the test line, the plurality of tone distinction markers, the identification marker on the manufacturing deviation, and the like included in the diagnostic kit 200.

The memory 120 may be stored with at least one instruction related to the electronic device 100. Further, the memory 120 may be stored with an operating system (O/S) for operating the electronic device 100. In addition, various software programs or applications for operating the electronic device 100 may be stored in the memory 120 according to the various embodiments of the disclosure. Further, the memory 120 may include a semiconductor memory such as a flash memory or a magnetic storage medium such as a hard disk.

The memory 120 may be stored with various software modules for operating the electronic device 100 according to the various embodiments of the disclosure, and the processor 130 may be configured to execute the various software modules stored in the memory 120 to control the operation of the electronic device 100. That is, the memory 120 may be accessed by the processor 130, and the reading/recording/modifying/updating and the like of data may be performed by the processor 130.

The term memory 120 may be used as a meaning including a memory 120, a read only memory (ROM, not shown) within the processor 130, a random access memory (RAM, not shown), or a memory card (not shown, e.g., micro SD card, memory stick) mounted to the electronic device 100.

According to the various embodiments of the disclosure, the memory 120 may be stored with first data for obtaining the biometric information measurement value according to the color value and second data for obtaining the diagnostic result according to the biometric information measurement value.

Specifically, the ‘first data’ may include information on a function or a lookup table capable of outputting the biometric information measurement value corresponding to the input color value, and the ‘second data’ may include information on a function or a lookup table capable of outputting the diagnostic result according to a section in which the input biometric information measurement value from among a plurality of pre-defined sections is included. The first data and the second data may be received from an external device together with an application implemented so as to perform the control method according to the disclosure and may be stored in the electronic device 100.

The memory 120 may be continuously stored with information on the biometric information measurement value and the diagnostic result obtained through the processor 130, and accordingly, the second data stored in the memory 120 in particular may be updated. In addition thereto, various information necessary within the scope of achieving the object of the disclosure may be stored in the memory 120.

The processor 130 may be configured to control the overall operation of the electronic device 100. Specifically, the processor 130 may be coupled with a configuration of the electronic device 100 which includes the camera 110 and the memory 120, and may control the overall operation of the electronic device 100 by executing at least one instruction stored in the memory 120 as described above.

The processor 130 may be implemented in various methods. For example, the processor 130 may be implemented as at least one from among an application specific integrated circuit (ASIC), an embedded processor, a microprocessor, a hardware control logic, a hardware finite state machine (FSM), or a digital signal processor (DSP). The term processor 130 in the disclosure may be used as a meaning which includes a central processing unit (CPU), a graphic processing unit (GPU), a main processing unit (MPU), and the like.

According to the various embodiments of the disclosure, the processor 130 may be configured to obtain, based on a user command for photographing the diagnostic kit 200 including at least one test line discolored according to the reaction between the sample collected from the body of the user and the reagent and the plurality of tone distinction markers having different tones from one another, the image on the diagnostic kit 200 through the camera 110, obtain the first color value on the at least one test line included in the obtained image and the second color value on each of the plurality of tone distinction markers included in the obtained image, obtain a first deviation information on the photographing environment of the image obtained based on the second color value, correct first data stored in the memory 120 based on the first deviation information, obtain the biometric information measurement value corresponding to the first color value based on the corrected first data, and obtain the diagnostic result corresponding to the biometric information measurement value obtained based on the second data stored in the memory 120.

Further, because the various embodiments according to the disclosure which is based on the control of the processor 130 has been described above with reference to FIGS. 1 to 9, redundant descriptions will be omitted.

The outputter 140 may include circuitry, and the processor 130 may be configured to output various functions which the electronic device 100 may perform through the outputter 140. The outputter 140 may include at least one from among a display 141, a speaker 142, and an indicator (not shown).

The display 141 may output image data under the control of the processor 130. Specifically, the display 141 may output an image pre-stored in the memory 120 under the control of the processor 130. The display 141 may be implemented as a liquid crystal display (LCD) panel, an organic light emitting diodes (OLED), or the like, and the display 141 may also be implemented as a flexible display, a transparent display, or the like according to circumstance. However, the display 141 according to the disclosure is not limited to a specific type.

The speaker 142 may output audio data under the control of the processor 130, and the indicator may be lighted under the control of the processor 130.

According to the various embodiments of the disclosure, the processor may be configured to provide the user with the diagnostic result through the outputter 140. Specifically, the processor 130 may be configured to visually display the obtained biometric information measurement value and the diagnostic result through the display 141, and specifically, control the display 141 to display a user interface including the obtained biometric information measurement value and the diagnostic result. In addition, the processor 130 may output the obtained biometric information measurement value and the diagnostic result through the speaker 142 in speech form.

The inputter 150 may include circuitry, and the processor 130 may be configured to receive a user command for controlling an operation of the electronic device 100 through the inputter 150. Specifically, the inputter 150 may be comprised of configurations such as a camera 110, a microphone (not shown), and a remote control signal receiver (not shown), or the like. The inputter 150 may also be implemented in a form included in the display 141 as a touch screen.

According to the various embodiments of the disclosure, the processor 130 may be configured to receive a user command for photographing the diagnostic kit 200 through the inputter 150, and may also receive a user command for searching or receiving the biometric information measurement value and the diagnostic result.

The communicator 160 may include circuitry, and perform communication with the external device. Specifically, the processor 130 may be configured to receive various data or information from the external device coupled through the communicator 160, and transmit various data or information to the external device.

The communicator 160 may include at least one from among a WiFi module, a Bluetooth module, a wireless communication module, and an NFC module. Specifically, each of the WiFi module and the Bluetooth module may perform communication through a WiFi method and a Bluetooth method. When using the WiFi module or the Bluetooth module, various connection information such as SSID may first be transmitted and received, and may transmit and receive various information after communicatively coupling using the connection information. In addition, the wireless communication module may perform communication according to various communication standards such as IEEE, Zigbee, 3rd generation (3G), 3rd generation partnership project (3GPP), long term evolution (LTE), 5th generation (5G), or the like. The NFC module may perform communication with a near field communication (NFC) method which uses a 13.56 MHz band from among the various RF-ID frequency bands such as, for example, and without limitation, 135 kHz, 13.56 MHz, 433 MHz, 860-960 MHz, 2.45 GHz, or the like.

According to the various embodiments of the disclosure, the processor 130 may be configured to receive information related to the first data and the second data from an external device through the communicator 160 and store in the memory 120. In addition, the processor 130 may transmit the biometric information measurement value and the diagnostic result obtained through the communicator 160 to the external device. The external device may be a user terminal such as a smart watch, and may be a server for providing health related information based on the received biometric information measurement value.

The image obtaining module 131 may refer to a module for obtaining the image on the diagnostic kit 200 through the camera 110. The image obtaining module 131 may receive an image signal obtained through the image sensor of the camera 110, and obtain an image (image data) corresponding to the received image signal.

The color value obtaining module 132 may refer to a module for obtaining the color value by analyzing the image on the diagnostic kit 200. Based on the image on the diagnostic kit 200 being received from the image obtaining module 131, the color value obtaining module 132 may obtain the first color value on the at least one test line included in the image on the diagnostic kit 200 and the second color value on each of the plurality of tone distinction markers included in the image on the diagnostic kit 200.

The environment deviation information obtaining module 133 may refer to a module for obtaining information (i.e., first deviation information) on the test line included in the image on the diagnostic kit 200 and environmental elements capable of influencing the color value of the tone supplementary marker. Specifically, based on the second color value on the plurality of tone supplementary markers being received from the color value obtaining module 132, the environment deviation information obtaining module 133 may obtain information on the color of the surrounding light source at the time of photographing the image based on the second color value, information on the light amount of the surrounding light source, and the like.

The manufacturing deviation information obtaining module 134 may refer to a module for obtaining information (i.e., second deviation information) on the manufacturing deviation which is apparent in the final product according to an error generated by a chemical/physical error which may be generated in the manufacturing process of the diagnostic kit 200. Specifically, when the image on the diagnostic kit 200 is received from the image obtaining module 131, the manufacturing deviation information obtaining module 134 may obtain information on a calibration curve of the diagnostic kit 200 based on the identification marker on the manufacturing deviation included in the image on the diagnostic kit 200.

The biometric information measurement value obtaining module 135 may refer to a module for obtaining the biometric information measurement value corresponding to the first color value based on information received from the environment deviation information obtaining module 133 and the manufacturing deviation information obtaining module 134. Specifically, the biometric information measurement value obtaining module 135 may correct the first data based on at least one from among the first deviation information received from the environment deviation information obtaining module 133 and the second deviation information obtained from the manufacturing deviation information obtaining module 134, and obtain the biometric information measurement value corresponding to the first color value based on the corrected first data. The biometric information measurement value obtaining module 135 may be configured so that information on the biometric information measurement value is provided to the user by transmitting the obtained biometric information measurement value to the outputter 140.

The diagnostic result obtaining module 136 may refer to a module for obtaining the diagnostic result corresponding thereto based on the biometric information measurement value received from the biometric information measurement value obtaining module 135. Specifically, when the biometric information measurement value is received from the biometric information measurement value obtaining module 135, the diagnostic result obtaining module 136 may obtain, based on which section from among the plurality of pre-defined numerical sections in the second data the biometric information measurement value belongs, the diagnostic result corresponding to the input biometric information measurement value. The diagnostic result obtaining module 136 may be configured to provide the diagnostic result to the user by transmitting information on the obtained diagnostic result to the outputter 140. Then, the diagnostic result obtaining module 136 may be able to transmit the biometric information measurement value and the information on the diagnostic result to the personalized data managing module 137.

The personalized data managing module 137 may refer to a module which personalizes the second data for each user based on the biometric information measurement value and the diagnostic result. Specifically, when information on the biometric information measurement value and the diagnostic result is received from the diagnostic result obtaining module 136, the personalized data managing module 137 may personalize the second data by storing in the memory 120 each time the biometric information measurement value and the diagnostic result on a user is obtained. As described above, the ‘personalization’ of the second data may refer to the range of second data used in finally obtaining the diagnostic result being extended and determined to be appropriate for the individual user as the biometric information measurement value per user and the diagnostic result are accumulatively stored.

The hardware configuration and the software module according to the disclosure has been described with reference to FIGS. 10 and 11 above, but this is merely one embodiment, and may be implemented in other methods than what was illustrated in FIGS. 10 and 11.

The control method of the electronic device 100 according to an embodiment described above may be implemented as a program and provided to the electronic device 100. Specifically, the program including the control method of the electronic device 100 may be stored and provided in a non-transitory computer readable medium.

Specifically, in terms of a computer readable recording medium including a program which executes the control method of the electronic device 100, the control method of the electronic device 100 may include obtaining, based on a user command for photographing the diagnostic kit including at least one test line which is discolored according to the reaction between the sample collected from the user body and the reagent and the plurality of tone distinction markers having different tones from one another, the image on the diagnostic kit through the camera included in the electronic device, obtaining the first color value on at least one test line included in the obtained image and the second color value on each of the plurality of tone distinction markers included in the obtained image, obtaining first deviation information on the photographing environment of the obtained image based on the second color value, correcting first data for obtaining the biometric information measurement value according to the first color value stored in the electronic device based on first deviation information, obtaining the biometric information measurement value corresponding to the first color value based on the corrected first data, and obtaining the diagnostic result corresponding to the biometric information measurement value obtained based on second data for obtaining the diagnostic result according to the biometric information measurement value stored in the electronic device.

The non-transitory computer readable medium may refer to a medium that stores data semi-permanently rather than storing data for a very short time, such as a register, a cache, or a memory 120, and is readable by a device. In detail, the aforementioned various applications or programs may be stored in the non-transitory computer readable medium, such as, for example, and without limitation, a compact disc (CD), a digital versatile disc (DVD), a hard disc, a Blu-ray disc, a universal serial bus (USB), a memory 120 card, a read only memory (ROM), and the like, and may be provided.

In the above, the control method of the electronic device 100, and the computer readable recording medium including the program which executes the control method of the electronic device 100 has been briefly described, but this is merely to omit redundant descriptions thereof, and the various embodiments with respect to the electronic device 100 may also be applied to the control method of the electronic device 100, and the computer readable recording medium including the program which executes the control method of the electronic device 100.

The machine-readable storage medium may be provided in the form of a non-transitory storage medium. Herein, “non-transitory” merely means that the storage medium is tangible and does not include a signal (e.g., electromagnetic wave), and the term does not differentiate data being semi-permanently stored in the storage medium and data temporarily being stored. For example, the ‘non-transitory storage medium’ may include a buffer in which data is temporarily stored.

According to an embodiment, a method according to one or more embodiments may be provided included in a computer program product. The computer program product may be exchanged between a seller and a purchaser as a commodity. The computer program product may be distributed in the form of a machine-readable storage medium (e.g., a compact disc read only memory (CD-ROM)), or distributed online through an application store (e.g., PLAYSTORE™) or directly between two user devices (e.g., smartphones). In the case of online distribution, at least a portion of the computer program product (e.g., downloadable app) may be at least stored temporarily in a storage medium such as a server of a manufacturer, a server of an application store, or a memory of a relay server, or temporarily generated.

According to one or more embodiments of the disclosure as described above, the electronic device 100 according to the disclosure may more accurately analyze the quantitative value on the color of the test line 211 apparent in the diagnostic kit 200 and provide the diagnostic result by taking into consideration information on the photographing environment of the image, information on the manufacturing deviation of the diagnostic kit 200, the intrinsic characteristic of the individual user with respect to the biometric information, and the like.

Each of the elements (e.g., a module or a program) according to the one or more embodiments of the disclosure as described above may be comprised of a single entity or a plurality of entities, and some sub-elements of the abovementioned sub-elements may be omitted, or different sub-elements may be further included in the various embodiments. Alternatively or additionally, some elements (e.g., modules or programs) may be integrated into one entity to perform the same or similar functions performed by each respective element prior to integration.

Operations performed by a module, program, or other element, in accordance with various embodiments, may be performed sequentially, in a parallel, repetitive, or heuristically manner, or at least some operations may be performed in a different order, omitted or a different operation may be added.

The terms “part” or “module” used in the disclosure may include a unit configured as a hardware, software, or firmware, and may be used interchangeably with terms such as, for example, and without limitation, logic, logic blocks, parts, circuits, or the like. “Part” or “module” may be a component integrally formed or a minimum unit or a part of the component performing one or more functions. For example, a module may be configured as an application-specific integrated circuit (ASIC).

One or more embodiments may be implemented with software including instructions stored in a machine-readable storage media (e.g., computer). The machine may call an instruction stored in the storage medium, and as a device capable of operating according to the called instruction, may include an electronic device (e.g., electronic device 100) according to embodiments.

Based on the instruction being executed by the processor, the processor may directly or under the control of the processor perform a function corresponding to the instruction using different elements. The instructions may include a code generated by a compiler or executed by an interpreter.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. An electronic device, comprising:
a camera;
a memory configured to store a first data to obtain a biometric information measurement value according to a color value and a second data to obtain a diagnostic result according to the biometric information measurement value; and
a processor configured to:
obtain an image of a diagnostic kit through the camera, the obtaining of the image based on receiving a user command for photographing the diagnostic kit, the diagnostic kit comprising at least one test line discolored according to a reaction between a sample collected from a body of a user and a reagent, and a plurality of tone distinction markers with tones different from one another,
obtain a first color value on the at least one test line comprised in the obtained image and a second color value on each of the plurality of tone distinction markers comprised in the obtained image,
obtain a first deviation information on a photographing environment of the obtained image based on the second color value,
correct the first data stored in the memory based on the first deviation information,
obtain the biometric information measurement value corresponding to the first color value based on the corrected first data, and
obtain a diagnostic result corresponding to the obtained biometric information measurement value based on the second data stored in the memory.

2. The electronic device of claim 1,
wherein the diagnostic kit further comprises an identification marker on a manufacturing deviation of the diagnostic kit, and
wherein the processor is further configured to:
obtain second deviation information on a manufacturing deviation of the diagnostic kit based on the identification marker comprised in the obtained image, and
correct the first data stored in the memory based on the first deviation information and the second deviation information.

3. The electronic device of claim 2,
wherein the first data comprises information on a function capable of outputting a biometric information measurement value corresponding to an input color value, and
wherein the processor is further configured to correct the first data by changing a coefficient of the function to a value corresponding to the first deviation information and the second deviation information.

4. The electronic device of claim 2,
wherein the plurality of tone distinction markers has the same color as a color of the at least one discolored test line,
wherein each of the plurality of tone distinction markers has different brightness or saturation from one another,
wherein the identification marker on the manufacturing deviation may be disposed on the diagnostic kit in a form of a barcode or a quick response (QR) code, and
wherein the processor is further configured to:
obtain a different second color value on each of the plurality of tone distinction markers based on the different brightness or saturation of each of the plurality of tone distinction markers comprised in the image,
obtain information on a calibration curve of the diagnostic kit based on the barcode or the QR code comprised in the image, and
obtain the second deviation information based on information on the calibration curve.

5. The electronic device of claim 1, wherein the second data comprises information on a function capable of outputting a diagnostic result according to a section, from among a plurality of pre-defined sections, in which the obtained biometric information measurement value is located.

6. The electronic device of claim 1, wherein the processor is further configured to:
update the second data stored in the memory based on the obtained biometric information measurement value and the obtained diagnostic result, and
obtain, based on obtaining a biometric information measurement value different from the biometric information measurement value after the second data is updated, a diagnostic result corresponding to the biometric information measurement value obtained after the second data is updated based on the updated second data.

7. The electronic device of claim 1,
wherein the at least one test line is a plurality of test lines corresponding to each of a plurality of testing items, and
wherein the processor is further configured to:
obtain a plurality of biometric information measurement values corresponding to each of the plurality of test lines, and
obtain a diagnostic result based on the plurality of biometric information measurement values.

8. The electronic device of claim 1,
wherein the diagnostic kit comprises a plurality of reagent pads comprising at least one test line,
wherein a plurality of tone supplementary markers is arranged between two reagent pads adjacent to each other from among the plurality of reagent pads, and
wherein the processor is further configured to obtain an image of the diagnostic kit comprising the plurality of reagent pads and the plurality of tone supplementary markers through the camera.

9. A control method of an electronic device, comprising:
obtaining an image of a diagnostic kit through a camera included in the electronic device, the obtaining of the image based on receiving a user command for photographing a diagnostic kit, the diagnostic kit comprising at least one test line discolored according to a reaction between a sample collected from a body of a user and a reagent, and a plurality of tone distinction markers with tones different from one another;
obtaining a first color value on the at least one test line comprised in the obtained image and a second color value on each of the plurality of tone distinction markers comprised in the obtained image;
obtaining a first deviation information on a photographing environment of the obtained image based on the second color value;
correcting a first data to obtain a biometric information measurement value according to the first color value stored in the electronic device based on the first deviation information;
obtaining the biometric information measurement value corresponding to the first color value based on the corrected first data; and
obtaining a diagnostic result corresponding to the obtained biometric information measurement value based on a second data for obtaining the diagnostic result according to the biometric information measurement value stored in the electronic device.

10. The method of claim 9,
wherein the diagnostic kit further comprises an identification marker on a manufacturing deviation of the diagnostic kit, and
wherein the correcting of the first data comprises:
obtaining a second deviation information on a manufacturing deviation of the diagnostic kit based on the identification marker comprised in the obtained image; and
correcting the first data stored in memory based on the first deviation information and the second deviation information.

11. The method of claim 10,
wherein the first data comprises information on a function capable of outputting a biometric information measurement value corresponding to an input color value, and
wherein the correcting the first data comprises correcting the first data by changing a coefficient of the function to a value corresponding to the first deviation information and the second deviation information.

12. The method of claim 10,
wherein the plurality of tone distinction markers has a color same as a color of the at least one discolored test line,
wherein each of the plurality of tone distinction markers has a brightness or saturation different from one another,
wherein the identification marker on the manufacturing deviation is disposed on the diagnostic kit in a form of a barcode or a quick response (QR) code,
wherein the obtaining the first color value and the second color value comprises obtaining a different second color value on each of the plurality of tone distinction markers based on the different brightness or saturation of each of the plurality of tone distinction markers comprised in the image, and
wherein the obtaining the second deviation information comprises:
obtaining information on a calibration curve of the diagnostic kit based on the barcode or the QR code comprised in the image; and
obtaining the second deviation information based on information on the calibration curve.

13. The method of claim 9, wherein the second data comprises information on a function capable of outputting a diagnostic result according to a section, from among a plurality of pre-defined sections, in which the obtained biometric information measurement value is located.

14. The method of claim 9, wherein the control method of the electronic device further comprises:
updating second data stored in memory based on the obtained biometric information measurement value and the obtained diagnostic result; and
obtaining, based on a biometric information measurement value different from the biometric information measurement value after the second data is updated being obtained, a diagnostic result corresponding to the biometric information measurement value obtained after the second data is updated based on the updated second data.

15. The method of claim 9, wherein the at least one test line comprises a plurality of test lines corresponding to each of a plurality of testing items,
wherein the control method of the electronic device comprises:
obtaining a plurality of biometric information measurement values corresponding to each of the plurality of test lines, and
obtaining a diagnostic result based on the plurality of biometric information measurement values.

16. The method of claim 9, wherein the diagnostic kit comprises a plurality of reagent pads comprising at least one test line,
wherein a plurality of tone supplementary markers is disposed between two reagent pads adjacent to each other from among the plurality of reagent pads, and
wherein the obtaining of the image off the diagnostic kit comprises obtaining the image of the diagnostic kit comprising the plurality of reagent pads and the plurality of tone supplementary markers through the camera.

17. A non-transitory computer readable recording medium comprising a program executing a control method of an electronic device, the method comprising:
obtaining an image of a diagnostic kit through a camera included in the electronic device, the obtaining of the image based on receiving a user command for photographing the diagnostic kit, the diagnostic kit comprising at least one test line discolored according to a reaction between a sample collected from a body of a user and a reagent, and a plurality of tone distinction markers with tones different from one another;
obtaining a first color value on the at least one test line comprised in the obtained image and a second color value on each of the plurality of tone distinction markers comprised in the obtained image;
obtaining a first deviation information on a photographing environment of the obtained image based on the second color value;
correcting a first data to obtain a biometric information measurement value according to the first color value stored in the electronic device based on the first deviation information;
obtaining the biometric information measurement value corresponding to the first color value based on the corrected first data; and
obtaining a diagnostic result corresponding to the obtained biometric information measurement value based on a second data for obtaining the diagnostic result according to the biometric information measurement value stored in the electronic device.

18. The non-transitory computer readable recording medium of claim 17, wherein a part of a reagent pad may be exposed outside of the diagnostic kit, the part of the reagent pad comprising the at least one test line discolored.

19. The non-transitory computer readable recording medium of claim 17, the method further comprising:
identifying a line which connects a center point of each of a plurality of tone supplementary markers included in the obtained image, and an angle between horizontal lines which divide the obtained image; and
when edges of the plurality of tone supplementary markers and edges of a reagent pad within the diagnostic kit in the obtained image are parallel with one another, identifying an interest area based on the identified angle and a distance between the plurality of tone supplementary markers and the reagent pad.
